(19) United States
(12) Patent Application Publication
(10) Pub. No.: US 2025/0346607 A1
(43) Pub. Date: Nov. 13, 2025

(54) METHOD FOR PREPARING A 1,4:3,6-DIANHYDROHEXITOL DIESTER COMPOSITION

(71) Applicant: ROQUETTE FRERES, LESTREM (FR)

(72) Inventors: Hélène AMEDRO, BETHUNE (FR); René SAINT-LOUP, Fontainebleau (FR); Théodore VANBESIEN, ARMENTIÈRES (FR)

(21) Appl. No.: 18/861,487

(22) PCT Filed: May 3, 2023
(86) PCT No.: PCT/EP2023/025204

§ 371 Date: Jul. 31, 2025

(30) Foreign Application Priority Data
May 3, 2022 (FR) FR2204207

(51) Int. Cl. C07D 493/04; C08K 5/00; C08K 5/09; C08K 5/101; C08K 5/103; C08K 5/1535

(52) U.S. Cl. CPC C07D 493/04; C08K 5/0016; C08K 5/09; C08K 5/101; C08K 5/103; C08K 5/1535

(57) ABSTRACT

The invention relates to a method for preparing a C13-C29 alkyl diester of 1,4:3,6-dianhydrohexitol, said method comprising:
a) a first step of esterification of 1,4:3,6-dianhydrohexitol with a fatty acid having a C13-C29 alkyl chain, said fatty acid being in excess, so as to form a reaction crude comprising a C13-C29 alkyl diester of 1,4:3,6-dianhydrohexitol and unreacted fatty acid;
b) a second step of esterification of the unreacted fatty acid with a primary or aromatic diol.

### TECHNICAL FIELD

The invention relates to a method for the preparation of a 1,4:3,6-dianedrohexitol alkyl diester composition and their use in the preparation of polycarbonate, in particular for improving the melt flow of polycarbonate.

### PRIOR ART

It is known that alkyl diesters of 1,4:3,6-dianedrohexitol such as isosorbide can be added to a polymer to facilitate its shaping. They then act as “plasticizers”.

The plasticizers most commonly used today belong to the phthalate ester family (phtalates). Phthalates are readily available on the market at low cost, but are being replaced because of toxicity concerns.

For example, patent EP 3 443 033 B1 describes that C8/C10 alkyl diesters of isosorbide, marketed by the applicant under the trade name Polysorb ID46, facilitate the preparation of polycarbonates, in particular by improving their melt flow.

1,4:3,6-dianedrohexitol alkyl diesters are conventionally prepared by esterification of a 1,4:3,6-dianedrohexitol with an excess fatty acid, typically in the presence of an acid catalyst. The reaction crude comprises unreacted fatty acid (residual fatty acid). As this fatty acid has a negative effect on the use of diester compositions as plasticizers, it is removed at the end of the esterification step by subjecting the reaction crude to a distillation step or a liquid-liquid washing step comprising washing with water in the presence of a weak base such as sodium bicarbonate, followed by a drying step.

As detailed in application WO 2006/103338 A1, certain 1,4:3,6-dianedrohexitol diester compositions exhibit an undesirable yellow coloration, particularly when these compositions are intended for use in transparent plastics such as polycarbonate. The solution proposed in this patent document is to treat the reaction crude with a particular acid, hypophosphorous acid, which acts as a decolorizing agent.

There is an ongoing need to develop new additives for the preparation of synthetic polymers.

In the course of its research, the Applicant Company found that in polycarbonate preparation methods, C8/C10 alkyl diesters did not have the expected effect, and in addition imparted coloration to the polycarbonate. It has developed new long-chain alkyl diesters of 1,4:3,6-dianhydrohexitols. During development, it was found that the application of a residual fatty acid removal step by liquid-liquid distillation or washing led to degradation of a fraction of the diester into a monoester, and yellowing of the product.

There is therefore a need to find a method making it possible to prepare on an industrial scale, with good yields, a composition of a long-chain alkyl diester of 1,4:3,6-1,4:3,6-dianhydrohexitol with low coloration and minimal residual fatty acid content, which can be used in the preparation of a polycarbonate, in particular during its shaping.

### Description

### First Aspect

According to a first aspect, the present invention relates to a method for preparing a composition of a C13-C29 alkyl diester of 1,4:3,6-dianedrohexitol, said method comprising:
a) a first step of esterification of 1,4:3,6-dianhydrohexitol with a fatty acid having a C13-C29 alkyl chain, said fatty acid being in excess, so as to form a reaction crude comprising a C13-C29 alkyl diester of 1,4:3,6-dianhydrohexitol and unreacted fatty acid;
b) a second step of esterification of the unreacted fatty acid with a primary or aromatic diol.

As previously mentioned, the applicant company has found that when a composition obtained by esterification of 1,4:3,6-dianedrohexitol with a long-chain fatty acid is purified by distillation, the resulting composition exhibits undesirable coloration. With regard to liquid-liquid washing, the applicant company has observed that the drying step, which is applied after washing with water in the presence of a weak base, generates coloration. It has also been found that in the case of saturated fatty acids, the resulting reaction crude is solid and can only be washed at temperatures above its melting point, typically above 80° C., resulting in partial hydrolysis of the 1,4:3,6-dianedrohexitol diester.

Without wishing to be bound by any particular theory, the inventors believe that in prior art methods, the risk of cleavage of the diester into a monoester increases with the temperature applied during removal by distillation or liquid-liquid washing, which is higher the longer the alkyl chain is.

The inventors have developed a method wherein, unlike prior art methods, the removal of residual fatty acid does not require liquid-liquid distillation or washing.

The method according to the invention is simple to implement on an industrial scale and cost-effective. It is based on two sequential esterification steps.

The first step comprises the esterification of 1,4:3,6-dianedrohexitol with a long-chain fatty acid, said fatty acid being in excess, so as to obtain as much diester as possible.

The second step involves esterifying the residual fatty acid with a primary or aromatic diol, so as to remove most, almost all, or even all of the fatty acid that has not reacted in the first esterification step.

This elimination by esterification has the advantage of being simpler to implement than elimination by distillation, and can be carried out directly in the esterification reactor used for the first esterification step.

The method according to the invention is therefore advantageously a single-stage method, i.e. a preparation method whose stages are carried out successively in the same reactor. The method according to the invention advantageously does not include an isolation, separation or purification step by evaporation, for example by distillation.

Furthermore, as will be detailed below, the compositions obtained via the method according to the invention exhibit low colorations and are effective in polycarbonate preparation methods.

“C13-C29 alkyl diester” means an alkyl diester of 1,4:3,6-dianedrohexitol wherein alkyl groups are bonded to the hydroxyl groups of 1,4:3,6-dianedrohexitol. This C13-C29 alkyl diester is produced by the esterification reaction of 1,4:3,6-dianhydrohexitol with a fatty acid. An esterification reaction can be written as follows: R—OH+HO(O)C—R′=>R—O(O)C—R′+H₂O. So, if the fatty acid used for esterification has a C13 alkyl chain R′, the alkyl group of the ester is R′ and is therefore a C13 alkyl group. If only one of the diol's two alcohol functions has been reacted by esterification, the ester is a monoester. It is a diester if the two alcohol functions of the diol have been reacted in an esterification reaction. The C13-C29 alkyl diester of 1,4:3,6-dianhydrohexitol may comprise different alkyl groups when the diester is obtained with 2 of the different fatty acids

For the purposes of the present invention, the expression “fatty acid with a C13-C29 alkyl chain” means a fatty acid R′-COOH comprising 14 to 30 carbon atoms, one carbon atom belonging to the carboxylic acid group (—COOH), the remainder of the carbon atoms belonging to a substituted or unsubstituted linear or branched alkyl chain comprising between 13 and 29 carbon atoms, or a mixture thereof. The alkyl chain is preferably saturated. The alkyl chain can be substituted with at least one group comprising a heteroatom selected from oxygen and nitrogen, preferably a ketone or hydroxyl group or an amine group. The chain can be substituted at the end of the alkyl chain by a cyclic group typically containing from 5 to 12 carbon atoms, preferably from 6 to 10 carbon atoms, preferably by a cyclohexane or cyclopentane ring, or by a C2-C4 alkyl chain linked to the fatty acid alkyl chain at two points of attachment so as to form an intra-chain ring, preferably an intra-chain cyclopropane.

### Step a)

In the first step, a 1,4:3,6-dianedrohexitol is esterified with a fatty acid having a C13-C29 alkyl chain.

1,4:3,6-dianydrohexitol

1,4:3,6-dianhydrohexitol is a diol with the empirical formula C₆H₁₀O₄.

The invention uses three isomers of 1,4:3,6-dianhydrohexitol: isosorbide, isomannide and isoidide, or mixtures thereof.

Isosorbide is preferred.

### Fatty Acids

The fatty acid preferably has a C13-C29, preferably C13-C17, alkyl chain.

By way of example, the fatty acid may be selected from stearic acid (octadecanoic acid C18:0), myristic acid (tetradecanoic acid C14:0), palmitic acid (hexadecanoic acid C16:0), isopalmitic acid (14-methylpentadecanoic acid), margaric acid (heptadecanoic acid C17:0), tuberculostearic acid (10-methylstearic acid), lactobacillic acid (10-[(1R,2S)-2-hexylcyclopropyl]decanoic acid), arachidic acid (icosanoic acid C20:0), phytanic acid (3,7,11,15-tetramethylhexadecanoic acid), 11-cyclohexylundecanoic acid (11-cyclohexylundecanoic acid), or a mixture thereof.

The fatty acid is advantageously a C16 or C18 fatty acid, or a mixture thereof.

Stearic acid and myristic acid are preferred.

### Excess Fatty Acids

In the first step, the fatty acid with a C13-C29 alkyl chain is in excess of the 1,4:3,6-dianedrohexitol in order to promote the formation of the C13-C29 alkyl diester of 1,4:3,6-dianedrohexitol and minimize the formation of the corresponding monoester.

“Excess” means a stoichiometric excess, that is an amount of fatty acid with a C13-C29 alkyl chain greater than the stoichiometric amount required for the esterification reaction with all the hydroxyl groups of the 1,4:3,6-dianedrohexitol present. In other words, in the first step of the method according to the invention, 2y+z moles of fatty acid are preferably reacted for y moles of 1,4:3,6-dianedrohexitol, with y and z in moles, 2y representing the stoichiometric amount of fatty acid with respect to y moles of 1,4:3,6-dianedrohexitol, and z representing the amount of fatty acid which is in excess with respect to said stoichiometric amount. The stoichiometric excess of fatty acid over 1,4:3,6-dianedrohexitol can also be expressed as a percentage using the formula (z/2y+z)×100.

The fatty acid is preferably in a stoichiometric excess of 10% to 100% with respect to 1,4:3,6-dianedrohexitol. Preferably, between 2.2 and 4 moles of fatty acid are reacted per mole of 1,4:3,6-dianhydrohexitol.

In other words, 1,4:3,6-dianedrohexitol is preferably reacted with fatty acid in a 1,4:3,6-dianedrohexitol/fatty acid molar ratio of between 1/2.2 and 1/4.

### Reaction Crude

For the purposes of the invention, “reaction crude” means the product of the first step of esterification of 1,4:3,6-dianedrohexitol with a fatty acid having a C13-C29 alkyl chain, and to which preferably no step of removal of residual fatty acids, in particular no step of isolation, separation or purification, in particular no step of removal of unreacted fatty acids by distillation or liquid-liquid route, has been applied.

The reaction crude preferably comprises an alkyl diester of 1,4:3,6-dianedrohexitol, an alkyl monoester of 1,4:3,6-dianedrohexitol, and unreacted fatty acid.

### Step b)

In the second step, the fatty acid that had not reacted in the first step is esterified with a primary or aromatic diol, in order to remove as much of this fatty acid as possible from the reaction crude.

The primary or aromatic diol has a higher reactivity than the 1,4:3,6-dianedrohexitol from step a). Preferably, it does not transesterify with the C13-C29 alkyl diester of 1,4:3,6-dianedrohexitol of the reaction crude obtained after step a).

### Primary or Aromatic Diol

“Diol” means a compound comprising two hydroxyl groups (—OH). The compound is preferably a hydrocarbon structure typically comprising between 2 and 20 carbon atoms, preferably between 2 and 12 carbon atoms.

The diol used in step b) is a primary or aromatic diol.

“Primary diol” means a diol whose hydroxyl groups are carried by a carbon atom also carrying at least 2 hydrogen atoms.

“Aromatic diol” means a diol whose hydroxyl groups are carried by a carbon atom of an aromatic ring.

“Aromatic ring” means a polyunsaturated cyclic hydrocarbon structure wherein the ring structure is planar and has (4n+2) delocalized electrons, n being an integer, having a single ring or several rings fused together and typically containing from 5 to 12 carbon atoms, preferably from 6 to 10 carbon atoms. A preferred aromatic ring is phenyl.

The primary diol preferably comprises from 2 to 20 carbon atoms, more preferably from 2 to 15 carbon atoms.

For example, the primary or aromatic diol may be selected from ethylene glycol, cyclohexanedimethanol (CHDM), neopentyl glycol (NPG), 1,4-butanediol, 1,2-benzenedimethanol, 1,3-benzenedimethanol, 1,4-benzenedimethanol, resorcinol, 1,5-pentanediol, 1,6 hexanediol, 1,8 octanediol, 1,10 decandiol, 1,12 dodecanediol, or mixtures thereof.

Ethylene glycol is preferred.

In the second step, the primary or aromatic diol is preferably introduced in stoichiometric proportions, or in slightly deficient proportions relative to the fatty acid that had not reacted in the first step, in order to eliminate virtually all of this fatty acid. Without wishing to be bound by any particular theory, the inventors assume that if the primary or aromatic diol is added in stoichiometric excess relative to the residual fatty acid, the final composition presents a risk of comprising the monoester of this primary or aromatic diol, which entails a risk of transesterification and therefore of generating 1,4:3,6-dianedrohexitol monoester.

“Primary or aromatic diol in slightly deficient proportions relative to the fatty acid that had not reacted in the first step” means a stoichiometric deficiency, that is a primary or aromatic diol quantity slightly below the stoichiometric quantity required for the esterification reaction of all the hydroxyl groups of the fatty acid that had not reacted in the first step.

The first esterification step generally involves reacting between 0.45 and 0.5 moles of primary or aromatic diol per mole of excess fatty acid. Typically, when the first esterification reaction of step a) is carried out with a molar amount of fatty acid that is in excess of the stoichiometric amount (which can be denoted “z”, as detailed above), the second esterification reaction of step b) is carried out with a molar amount of primary diol that corresponds to between 45% and 50% of the molar amount of fatty acid used in excess of the stoichiometric amount in step a). By way of example, if in step a) 2.3 moles of fatty acid are introduced per mole of 1,4:3,6-dianedrohexitol (i.e. an excess of z=0.3 moles of fatty acid) then in step b) preferably between 0.3×45%=0.135 and 0.3×50% (i.e. 0.3/2)=0.15 mol primary or aromatic diol.

### Esterification Conditions

The esterification steps can be carried out under the conventional conditions already used in the literature. These esterification methods are described, for example, in documents WO 99/45060 A1 or WO 2006/103338 A1.

The esterification steps are preferably carried out in the presence of at least one acid catalyst.

The acid catalyst used can be of a very varied nature, for example an acid catalyst chosen from hypophosphorous acid, hydrochloric acid, sulfuric acid, para-toluene sulfonic acid (APTS), methanesulfonic acid (AMS), trifluoromethanesulfonic acid, trifluoroacetic acid, trichloroacetic acid, tin ethyl-2-hexanoate, phosphotungstic acid and silicotungstic acid or a mixture of these acids or a macroporous or non-macroporous resin comprising at least one of these acids.

In the case of acid catalyst mixtures, they may or may not be introduced into the reaction medium simultaneously.

The mass quantity of acid catalyst can range from 0.05 to 20% of the mass of 1,4:3,6-dianhydrohexitol introduced into the reactor, for example from 0.1 to 10%.

It should be noted that hypophosphorous acid can also act as a color reducer. This may be introduced into the reaction medium simultaneously, or not, with another acid catalyst and/or the fatty acid. According to one variant, this introduction is carried out before or at the start of the esterification reaction, i.e. before the introduction of the acid catalyst and/or fatty acid. Advantageously, and regardless of when it is introduced, hypophosphorous acid is introduced in an amount of between 0.05 and 2%, preferably between 0.1 and 1%, expressed on a dry weight basis relative to the dry weight of 1,4:3,6-dianedrohexitol(s) used. According to another variant, hypophosphorous acid is introduced, whether or not simultaneously with the acid esterification catalyst, in a hypophosphorous acid/acid catalyst ratio of less than 1/1, said ratio being expressed as dry weight of hypophosphorous acid relative to the dry weight of acid catalyst. In particular, said ratio can be between 0.01/1 and 0.9/1, preferably between 0.02/1 and 0.8/1. When the catalyst is APTS, methanesulfonic acid, or phosphotungstic acid, said ratio can advantageously be between 0.05/1 and 0.4/1.

The temperature in the reactor can range from 90 to 200° C., generally from 100 to 160° C. To carry out the esterification reaction, water is generally removed, for example by distilling the reaction medium, in order to promote diester formation. To facilitate this removal, the reaction medium can be placed under vacuum, for example at a level ranging from 10 to 200 mbar. Reaction conditions such as vacuum level and temperature can be varied during the reaction.

The first esterification step usually lasts as long as it takes to achieve satisfactory conversion to 1,4:3,6-dianhydrohexitol diester. This can vary widely, from 1 to 10 hours.

The second esterification stage generally lasts until the free fatty acid has been satisfactorily eliminated. This can vary widely, from 1 to 10 hours.

A neutralization step can also be carried out on the catalyst introduced, by introducing a base, such as soda ash, in molar quantities equivalent to the molar quantities of catalyst introduced. This neutralization step is preferably carried out after the second esterification step.

### Color Reducer

The method according to the invention can also include a step for decolorizing the reaction crude from step a) using color reducers, such as activated carbon or hydrogen peroxide, or bleaching earths such as bleaching clay, bentonite, or montmorillonite.

This step can be carried out successively following one of steps a) and/or b). In this embodiment, the decolorizing agents are added after one of steps a) and/or b).

This step can also be carried out simultaneously with steps a) and/or b). In this embodiment, the decolorizing agents are added after the start of one of steps a) and/or b).

Treatment with activated carbon is carried out, for example, by contacting the reaction crude with 1 to 5% by weight of activated carbon. The temperature during this treatment can be around 100° C. The duration is generally several tens of minutes, for example around one hour. At the end of the treatment, the activated carbon is separated by filtration. Treatment with bleaching earth is similar to treatment with activated carbon.

A conventional hydrogen peroxide decolorizing treatment consists, for example, in introducing 0.5 to 2% 100% hydrogen peroxide into the composition to be decolorized, over a period ranging from 30 to 60 minutes, at a temperature of between 90°° C. and 100° C., followed by agitation of the composition for one to two hours at this temperature. When it is desired to combine these two types of decolorizing treatment, hydrogen peroxide treatment is preferred over activated carbon treatment.

Preferred embodiment

In a preferred embodiment, the method according to the invention enables the preparation of a composition of a C13-C29 alkyl diester of isosorbide, said method comprising:
a) a first step of esterification of isosorbide with a fatty acid having a C13-C29 alkyl chain in excess, for example in an isosorbide/fatty acid molar ratio of between 1/2.3 and 1/3, so as to form a reaction crude comprising an alkyl diester of isosorbide and the unreacted fatty acid;
b) a second step of esterification of the unreacted fatty acid with a primary or aromatic diol, preferably ethylene glycol,
wherein the fatty acid with a C13-C29 alkyl chain is preferably a fatty acid with a C13, C14, C15, C16, C17 or C18 alkyl chain.

### Second Aspect

A second object of the invention relates to a C13-C29 alkyl diester composition of 1,4:3,6-dianedrohexitol obtainable by the method of the first object of the invention.

Indeed, when the diester composition is obtained from different 1,4:3,6-dianedrohexitols, different primary and/or aromatic diols, and/or different fatty acids with a C13-C29 alkyl chain, very complex and diverse compositions are obtained which can only be defined more satisfactorily by the preparation method.

However, compositions according to invention can be defined relatively easily when using a limited number of 1,4:3,6-dianedrohexitol(s), primary and/or aromatic diol(s), and/or fatty acid(s) with a C13-C29 alkyl chain.

### Third Aspect

Thus, a third object of the invention relates to a C13-C29 alkyl diester composition comprising, by weight of the composition:
35 to 85% a C13-C29 alkyl diester of 1,4:3,6-dianedrohexitol (A),
10 to 50% diester of a primary or aromatic diol (B),
less than 6% C13-C29 alkyl monoester of 1,4:3,6-dianedrohexitol (C),
less than 3% fatty acid with a C13-C29 alkyl chain (D),
   the total content of C13-C29 alkyl diester of 1,4:3,6-dianedrohexitol (A) and diester of a primary or aromatic diol (B) being between 80% and 99%, preferably between 90% and 99%.
   the total content of C13-C29 alkyl diester of 1,4:3,6-dianedrohexitol (A) and diester of a primary or aromatic diol (B) being between 80% and 99%, preferably between 90% and 99%.

The 1,4:3,6-dianedrohexitol, the primary or aromatic diol, the fatty acid and the esters are as defined in the first object of the invention.

The composition according to the invention has the advantage of being lightly colored.

The applicant company has also discovered that, surprisingly, compositions comprising a mixture of a C13-C29 alkyl diester of 1,4:3,6-dianedrohexitol and a diester of a primary or aromatic diol exhibit equivalent or even superior performance to compositions obtained by removing residual fatty acid in accordance with prior art methods, by distillation or liquid-liquid washing, and thus comprising only one or more alkyl diesters of 1,4:3,6-dianedrohexitol, but no diester of a primary or aromatic diol.

The composition according to the invention preferably comprises 50 to 85% diester (A).

The composition according to the invention preferably comprises from 10 to 50%, more preferably from 10 to 35% diester (B).

The composition according to the invention preferably comprises less than 5%, 4%, 3%, or less than 2% monoester (C), typically from 0.5 to 6%, even more typically from 2 to 6% monoester (C).

The composition according to the invention preferably comprises less than 2% or 1% fatty acid (D), typically from 0.5 to 3%, even more typically from 1 to 3% fatty acid (D).

The 1,4:3,6-dianedrohexitol is preferably isosorbide.

Preferably, the alkyl diester is C13-C17 and the fatty acid has a C13-C17 alkyl chain.

The primary or aromatic diol is preferably ethylene glycol.

In a preferred embodiment, the composition according to the invention comprises, by weight of composition:
35 to 85% isosorbide distearate (A),
10 to 50% ethylene glycol distearate (B),
less than 6% isosorbide monostearate (C),
less than 3% stearic acid.

In a preferred embodiment, the composition according to the invention comprises, by weight of composition:
35 to 85% isosorbide dimyristate (A),
10 to 50% ethylene glycol dimyristate (B),
less than 6% isosorbide monomyristate (C),
less than 3% myristic acid.

The relative quantities of the various esters and fatty acids can be determined by standard methods known to the person skilled in the art, typically by gas chromatography or HPLC.

The composition may also comprise traces of a monoester of a primary or aromatic diol (E), for example from 0 to 1%, preferably from 0 to 0.1%, of monoester (E), by weight of the composition.

The composition may also include traces, for example from 0 to 1%, preferably from 0 to 0.1%, even more preferably from 0 to 0.01% of primary or aromatic diol (F), by weight of the composition.

The sum of the contents of esters (A), (B), (C), (E), fatty acids (D) and primary or aromatic diol (F) is preferably 100%.

### APHA Coloration

The composition advantageously has an APHA color index of less than 30.

The APHA color index of the composition according to the invention is preferably between 10 and 30, more preferably between 15 and 25, even more preferably between 18 and 22.

This is particularly advantageous for use of the composition according to the invention as an additive, typically as a flow agent in the manufacture of a transparent synthetic polymer such as a polycarbonate.

In the present invention, the APHA color index is measured directly on a sample of the composition according to the procedure in ASTM D8005-18 of March 2018. It should be noted that some compositions may be in the form of a solid with a melting point of around 80° C. In this case, the color index is measured in accordance with ASTM D8005-18 of March 2018, except that the measurement is carried out at a temperature of 90° C. (i.e. above the melting point of the composition) so that the composition is in liquid form.

### Fourth Aspect

A third object of the invention concerns the use of a composition according to the second or third object of the invention in a method for preparing a synthetic polymer.

The composition according to the second or third object of the invention is preferably used as a plasticizer.

By “plasticizer” we generally mean a product which, when mixed in sufficient quantity with a polymer, facilitates its shaping, for example by lowering the glass transition temperature of said polymer.

The composition according to the second or third object of the invention is particularly suitable for the preparation of transparent synthetic polymers, wherein color is an issue, such as polycarbonate.

The composition according to the second or third object of the invention is preferably used in a method for preparing a polycarbonate, in particular during the shaping of a polycarbonate, to improve melt flow.

The method for preparing polycarbonate comprises at least one step of shaping a polycarbonate by extruding a mixture comprising a diester composition according to the second or third object of the invention with a polycarbonate, the mixture typically containing between 3 and 5% by weight of composition according to the second or third object of the invention.

The polycarbonate can be an aliphatic polycarbonate or an aromatic polycarbonate. By way of example, the polycarbonate can be an aromatic polycarbonate typically such as those described in patent EP 3 443 033 B1. The aromatic polycarbonate is preferably an aromatic polycarbonate containing monomer units of the formula —[CO—O-pR1-R2-pR1-O]ₙ, wherein R1 represents phenyl optionally substituted by C1-C4 alkyl; R2 represents a group R3(R4R5), with R3 being a carbon atom or a ring with 6 carbon atoms and R4 and R5, which may be identical or different from one another, each represent a hydrogen atom or a group (R6)n, wherein R6 represents C1-C4 alkyl, n representing 1, 2 or 3.

### EXAMPLES

### Analytical Methods

### Measurement of Ester and Fatty Acid Mass Quantities

The relative proportions of each ester are measured by gas chromatography on a Varian 3400 with FID detection and type 1077 split/splitless injector. The column used is a DB1 from J & W Scientific, 30 meters long, 0.32 mm internal diameter, 0.25 mm film thickness. Temperature conditions are: injector and detector: 300° C.; column: programmed from 100° C. to 320° C. at 7° C./min, held for 15 min at 320° C. Injection is carried out in split mode at 80 ml/min, with a column head pressure of 14 psi and helium as the carrier gas.

The relative amount of diester is given by the ratio of the sum of the areas of the compounds corresponding to isosorbide diesters to an outside standard, namely heptadecanoic acid.

### APHA Color Measurement

The APHA color index is measured according to ASTM D8005-18, March 2018.

### Example 1: Synthesis of Isosorbide Distearate Followed by Esterification of Residual Fatty Acids by Addition of Ethylene Glycol (As Per the Invention)

In a 2 L jacketed reactor, 150 g of isosorbide (1 eq) and 876 g of stearic acid (C18:0, 3 eq) were introduced. Under a nitrogen sweep, the reactor was heated to 90° C. to melt the medium. When the medium was melted, a stain was taken (APHA=17). The following were then introduced, still under nitrogen: 3% methanesulfonic acid by mass (relative to isosorbide), 5% hypophosphorous acid by mass (relative to isosorbide) and 1% powdered activated carbon by mass (relative to the total mass of input). The medium was heated to 160° C. and a vacuum ramp was then applied, from 100 mbar to 5 mbar over 1 hour.

After 2 hours, the medium was cooled to 100° C. under nitrogen flow. A sample was taken for analysis, showing the presence of 34% fatty acid, 2% isosorbide monoester and 64% isosorbide diester.

0.5 eq ethylene glycol (relative to isosorbide) is then added to the medium. The temperature was reduced to 180° C. and the medium was left to stir for a further 2 hours under a vacuum of 5 mbars.

The medium was then cooled to 100° C. and returned to nitrogen, and a stoichiometric amount of sodium hydroxide was added to neutralize the AMS and hypophosphorous acid. The reaction medium is hot-filtered (100° C.) on a Beko KD3 filter. The results are as follows (relative % in the composition compared with the total weight of the composition):

| Isosorbide | EG | Isosorbide | EG | Residual | |
|---|---|---|---|---|---|
| diester | diester | monoester | monoester | fatty acid | APHA |
| (%) | (%) | (%) | (%) | (%) | Coloration |
| 57 | 34 | 5.9 | 0.1 | 3 | 19 |

### Example 2: Synthesis of Isosorbide Distearate and Purification by Distillation (Comparative)

In a 2 L jacketed reactor, 150 g of isosorbide (1 eq) and 876 g of stearic acid (C18, 3 eq) were introduced. Under a nitrogen sweep, the reactor was heated to 90° C. to melt the medium. Once the medium has melted, a stain is taken (APHA=19). Then, still under nitrogen, 3% methanesulfonic acid by mass (relative to isosorbide), 5% hypophosphorous acid by mass (relative to isosorbide) and 1% powdered activated carbon by mass were introduced. (relative to the total mass of inputs) The medium was heated to 160° C. and a vacuum ramp was applied, from 100 mbar to 5 mbar over 2 hours, to distill the water of reaction. The medium was then cooled to 100° C. and a stoichiometric amount of sodium hydroxide added to neutralize AMS and hypophosphorous acid. The reaction medium was hot-filtered (100° C.) on a Beko KD3 filter. Still at 100° C., 3000 ppm Irganox 1010 was added.

The reaction crude was distilled on a wiped-film evaporator (distillation area: 0.0045 m²) at 190° C. under vacuum at 0.1 mbars and at a feed rate of 1 Kg/h to distill off the excess fatty acid. The composition of the diester in the residue is as follows (relative % in the composition with respect to the total weight of the composition):

| Isosorbide | Monoester | Residual fatty | APHA |
|---|---|---|---|
| diester (%) | isosorbide (%) | acid (%) | Coloration |
| 93 | 3 | 4 | 85 |

### Example 3: Varying the Fatty Acid Excess of Example 1 (As Per the Invention)

In a 2 L jacketed reactor, 150 g of isosorbide (1 eq) and 671 g of stearic acid (C18, 2.3 eq) were introduced. Under a nitrogen sweep, the reactor was heated to 90° C. to melt the medium. Once the medium has melted, a stain is taken (APHA=19). The following were then introduced, still under nitrogen: 3% methanesulfonic acid by mass (relative to isosorbide), 5% hypophosphorous acid by mass (relative to isosorbide) and 1% powdered activated carbon by mass (relative to the total mass of input). The medium was heated to 160° C. and a vacuum ramp was then applied, from 100 mbar to 65 mbar over 2 hours.

After 2 hours, the medium was cooled to 100° C. under nitrogen flow. 0.15 eq ethylene glycol (relative to the isosorbide) was then added to the medium. The temperature was reduced to 180° C. and the medium left to stir for a further 2 hours.

The medium was then cooled to 100° C. and a stoichiometric amount of sodium hydroxide added to neutralize AMS and hypophosphorous acid. The reaction medium is hot-filtered (100° C.) on a Beko KD3 filter. The results are as follows (relative % in the composition compared with the total weight of the composition):

| | Ethylene | | Ethylene | | |
|---|---|---|---|---|---|
| Isosorbide | glycol | Isosorbide | glycol | Residual | |
| diester | diester | monoester | monoester | fatty acid | APHA |
| (%) | (%) | (%) | (%) | (%) | Coloration |
| 83 | 13 | 1.9 | 0.1 | 1 | 21 |

### Example 4: Direct Incorporation of the Second Diol, Non-Sequential Esterification (Comparative)

In a 2 L jacketed reactor, 150 g isosorbide (1 eq) is introduced along with 671 g stearic acid (C18, 2.3 eq) and 0.15 eq ethylene glycol. Under a nitrogen sweep, the reactor was heated to 90° C. to melt the medium. When the medium had melted, a stain was taken (APHA=19). Then, still under nitrogen, 3% methanesulfonic acid by mass (relative to isosorbide), 5% hypophosphorous acid by mass (relative to isosorbide) and 1% m (relative to total input mass) of powdered activated carbon were introduced. The medium was heated to 160° C. and a vacuum ramp was then applied, from 100 mbar to 5 mbar over 4 hours.

The medium was then cooled to 100° C. and a stoichiometric amount of sodium hydroxide added to neutralize AMS and hypophosphorous acid. The reaction medium was hot-filtered (100° C.) on a Beko KD3 filter to give the following results (relative % of the composition):

| Isosorbide | EG | Isosorbide | | Residual | |
|---|---|---|---|---|---|
| diester | diester | monoester | EG | fatty | APHA |
| (%) | (%) | (%) | monoester | acid (%) | Coloration |
| 74 | 13 | 7 | <0.1 | 6 | 19 |

Direct incorporation of the second diol at the start is accompanied by an increase in monoesters and a higher residual fatty acid content.

### Example 5: Synthesis of an Isosorbide Myristate Composition (As Per the Invention)

Example 1 has been reproduced, replacing stearic acid with myristic acid. (C14:0). The results are as follows:

The composition of the medium is as follows (relative % in the composition with respect to the total weight of the composition):

| Isosorbide | EG | Isosorbide | | Residual | |
|---|---|---|---|---|---|
| diester | diester | monoester | EG | fatty | APHA |
| (%) | (%) | (%) | monoester | acid (%) | Coloration |
| 84 | 13 | 1 | <0.1 | 2 | 19 |

### Example 6: Synthesis of Isosorbide Distearate and Purification by Distillation (Comparative)

A comparative example identical to Example 2 was produced, except that stearic acid (C18:0) is replaced by myristic acid (C14:0). The results after distillation are as follows (relative % in the composition compared with the total weight of the composition):

| | Monoester | Residual | APHA |
|---|---|---|---|
| Diester (%) | (%) | fatty acid (%) | Coloration |
| 93 | 5 | 2 | 77 |

### Example 7: (As Per the Invention)

Example 2 was reproduced by replacing ethylene glycol with cyclohexanedimethanol (CHDM). The results are as follows (relative % in the composition compared with the total weight of the composition)

| Isosorbide | | Isosorbide | | Residual | |
|---|---|---|---|---|---|
| diester | CHDM | monoester | CHDM | fatty | APHA |
| (%) | diester | (%) | monoester | acid (%) | Coloration |
| 80 | 11 | 5 | 1 | 3 | 20 |

### Example 8 (As Per the Invention)

Example 2 was reproduced by replacing ethylene glycol with neopentylglycol (NPG). The results are as follows (relative % in the composition compared with the total weight of the composition):

| | NPG | | | Residual | |
|---|---|---|---|---|---|
| Diester | diester | Monoester | NPG | fatty | APHA |
| (%) | (%) | (%) | monoester | acid (%) | Coloration |
| 84 | 14 | 1 | <0.1 | 1 | 19 |

### Example 9 (As Per the Invention)

Example 2 was reproduced by replacing ethylene glycol with 1,4-butanediol. The results are as follows (relative % in the composition compared with the total weight of the composition):

| | 1,4- | | | | |
|---|---|---|---|---|---|
| | BDO | | BDO | Residual | |
| Diester | diester | Monoester | monoester | fatty | APHA |
| (%) | (%) | (%) | (%) | acid (%) | Coloration |
| 82 | 14 | 2 | <0.1 | 2 | 19 |

### Example 10 (As Per the Invention)

Example 2 was reproduced by replacing ethylene glycol with 1,4-benzenedimethanol. The results are as follows (relative % in the composition compared with the total weight of the composition):

| | 1,4- | | | | |
|---|---|---|---|---|---|
| | BDM | | 1,4- | Residual | |
| Diester | diester | Monoester | BDM | fatty | APHA |
| (%) | (%) | (%) | monoester | acid (%) | Coloration |
| 82 | 14 | 2 | <0.1 | 2 | 25 |

### Example 11 (As Per the Invention)

Example 2 was reproduced by replacing ethylene glycol with resorcinol. The results are as follows (relative % in the composition compared with the total weight of the composition):

| | Resorcinol | | | Residual | |
|---|---|---|---|---|---|
| Diester | diester | Monoester | Resorcinol | fatty | APHA |
| (%) | (%) | (%) | monoester | acid (%) | Coloration |
| 81 | 11 | 4 | 1 | 3 | 25 |

### Example 12: Use in a Method for Preparing Polycarbonate (As Per the Invention)

The diester compositions of Examples 1, 3, 5 and 7 to 11 (AS PER THE INVENTION) have been used in a method for preparing polycarbonate.

A mixture comprising polycarbonate and about 0.5% of a diester composition, % by weight of mixture, was extruded.

The compositions of Examples 1, 3, 5 and 7 to 11 (AS PER THE INVENTION) have proved effective in improving the melt flow of polycarbonate. By way of example: In the case of the composition from example 1 and 3, the melt flow index (MFI) was determined in accordance with ISO 1133 on a dedicated instrument (CEAST brand, model 7024). Non-additivated extruded polycarbonate has an MFI of 12.9 g/10 min. The same polycarbonate additivated with 0.5% of the composition from Example 1 has an MFI of 43.5 g/10 min. The same polycarbonate additivated with 0.5% of the composition from Example 3 has an MFI of 42.75 g/10 min.

Furthermore, no yellowing of the final product was observed.

### Example 13: Use in a Method for Preparing Polycarbonate (Comparative)

A composition of C8/C10 alkyl diesters of isosorbide marketed by the applicant company under the name Polysorb ID46 has been used in a polycarbonate preparation method.

The composition of Polysorb ID46 did not improve the melt flow of the polycarbonate. It seems that diesters are vaporized during shaping due to the short alkyl chains. For example, non-additivated extruded polycarbonate has an MFI of 12.9 g/10 min. The same polycarbonate additivated with 0.5% Polysorb ID46 has an MFI of 15.7 g/10 min.

The polycarbonate obtained after extrusion has an undesirable yellow color.

1. A method for preparing a C13-C29 alkyl diester composition of 1,4:3,6-dianedrohexitol, said method comprising:
a) a first step of esterification of 1,4:3,6-dianhydrohexitol with a fatty acid having a C13-C29 alkyl chain, said fatty acid being in excess, so as to form a reaction crude comprising a C13-C29 alkyl diester of 1,4:3,6-dianhydrohexitol and unreacted fatty acid;
b) a second step of esterification of the unreacted fatty acid with a primary or aromatic diol.

2. The method according to claim 1, wherein the 1,4:3,6-dianhydrohexitol is selected from isosorbide, isomannide, isoidide, or a mixture thereof.

3. The method according to claim 1, wherein the fatty acid is selected from stearic acid, myristic acid, palmitic acid, isopalmitic acid, margaric acid, tuberculostearic acid, lactobacillic acid, arachidic acid, phytanic acid, chaulmoogric acid, 11-cyclohexylundecanoic acid or a mixture thereof.

4. The method according to claim 1, wherein the primary or aromatic diol is selected from ethylene glycol, cyclohexanedimethanol (CHDM), neopentylglycol (NPG), 1,4-butanediol, 1,4-benzenedimethanol, resorcinol, 1,5-pentanediol, 1,6 hexanediol, 1,8 octanediol, 1,10 decandiol, 1,12 dodecanediol or a mixture thereof.

5. The method according to claim 1, wherein the first and second esterification steps are carried out in the presence of an acid catalyst.

6. A C13-C29 alkyl diester composition of 1,4:3,6-dianedrohexitol obtainable by the method of claim 1.

7. A C13-C29 alkyl diester composition of 1,4:3,6-dianedrohexitol comprising, by weight of the composition:
35 to 90% a C13-C29 alkyl diester of 1,4:3,6-dianedrohexitol (A),
10 to 50% diester of a primary or aromatic diol (B),
less than 6% C13-C29 alkyl monoester of 1,4:3,6-dianedrohexitol (C),
less than 3% fatty acid with a C13-C29 alkyl chain (D),
the total content of C13-C29 alkyl diester of 1,4:3,6-dianedrohexitol (A) and diester of a primary or aromatic diol (B) being between 80% and 99%, preferably between 90% and 99%.

8. The composition according to claim 6, wherein the 1,4:3,6-dianydrohexitol is isosorbide.

9. The composition according to claim 6, wherein the alkyl diester is C13-C17 and the fatty acid has a C13-C17 alkyl chain.

10. The composition according to claim 6, wherein the primary or aromatic diol is ethylene glycol.

11. The composition according to claim 6, comprising, by weight of the composition:
35 to 90% isosorbide distearate (A),
10 to 50% ethylene glycol distearate (B),
less than 6% isosorbide monostearate (C),
less than 3% stearic acid (D).

12. The composition according to claim 6 comprising, by weight of the composition:
35 to 90% isosorbide dimyristate (A),
10 to 50% ethylene glycol dimyristate (B),
less than 6% isosorbide monomyristate (C),
less than 3% myristic acid (D).

13. The composition according to claim 6, which has an APHA color index of less than 30.

14. A use of a composition according to claim 6 in a method for preparing a polycarbonate, in particular during the shaping thereof, to improve the melt flow of a polycarbonate.

15. The composition according to claim 7, wherein the 1,4:3,6-dianydrohexitol is isosorbide.

16. The composition according to claim 7, wherein the alkyl diester is C13-C17 and the fatty acid has a C13-C17 alkyl chain.

17. The composition according to claim 7, wherein the primary or aromatic diol is ethylene glycol.

18. The composition according to claim 7 comprising, by weight of the composition:
35 to 90% isosorbide distearate (A),
10 to 50% ethylene glycol distearate (B),
less than 6% isosorbide monostearate (C),
less than 3% stearic acid (D).

19. The composition according to claim 7 comprising, by weight of the composition:
35 to 90% isosorbide dimyristate (A),
10 to 50% ethylene glycol dimyristate (B),
less than 6% isosorbide monomyristate (C),
less than 3% myristic acid (D).
